(19) United States
(12) Patent Application Publication
(10) Pub. No.: US 2016/0000973 A1
(43) Pub. Date: Jan. 7, 2016

(54) REPORTER SCAFFOLDS

(71) Applicant: CASE WESTERN RESERVE UNIVERSITY, Cleveland, OH (US)

(72) Inventor: Erin Lavik, Cleveland Heights, OH (US)

(21) Appl. No.: 14/793,202

(22) Filed: Jul. 7, 2015

Related U.S. Application Data
(60) Provisional application No. 62/021,367, filed on Jul. 7, 2014.

(51) Int. Cl. A61L 27/50; A61L 27/44

(52) U.S. Cl. CPC A61L 27/50; A61L 27/44

(57) ABSTRACT

The invention provides for the development of reporter scaffolds comprising sustained release reporter molecules and methods of using these scaffolds to administer therapeutic agent and/or monitor the effect of the transplant on the surrounding tissue or monitoring the status or condition of transplanted cells over time after the scaffold is in place.

### FIELD OF INVENTION

The invention provides for the development of reporter scaffolds and methods of using these scaffolds to administer therapeutic agents and/or to support the transplantation of cells as a therapy. The reporter scaffold releases a reporter molecule that allows for monitoring cellular response to the therapeutic agents and/or the behavior of the transplanted cells. The invention also provide for methods of monitoring cellular responses and function over time after the scaffold is in place, wherein the scaffold comprises a slow release reporter protein that can be visually detected.

### BACKGROUND

Cellular transplantation strategies have shown signs of success both in animals and the clinic. One of the fundamental challenges, is determining the status of cells after administration. Scaffolds have been used to facilitate the transplantation of cells in the various organs, such as the eye. For example, Scaffolds are often used in conjunction with cellular transplantation in the eye to improve survival of the cells, to promote specific differentiation of cells, and to direct cellular architecture such as the alignment of photoreceptors and bipolar cells [5, 6]. The use of a scaffold can improve cell survival as well as provide a matrix to guide the organization of cells. Up to this point, however, the scaffolds have not provided any feedback on the status of cells grown on them. The majority of materials used as scaffolds such as the degradable polyesters and hydrogel systems in the eye lend themselves to presenting reporter molecules to cells over time [4, 7, 8]. The present invention goes beyond using these scaffolds as supports to reporting on the state of the transplanted cells by combining them with the reporter molecules.

While one can genetically engineer cells to express reporter molecules, this manipulation can limit subsequent clinical translation. Therefore, non-genetic reporter approaches are preferable, such as florescent reporter molecules. The present invention provide for methods of understanding the behavior of transplanted cells in vivo more broadly.

### SUMMARY OF INVENTION

The present invention loads polymer scaffolds with fluorescent reporter dyes that report on cellular response/behavior such as oxidative stress, intracellular pH, and the live/dead assay molecules. The polymers will deliver the reporter molecules over time. These dyes could be added directly to the cells, but they wash out or are diluted over time, and in the case of viability dyes, they are only incorporated if the cells are dying. Therefore, the dye needs to be added regularly to be able to investigate the cell behavior. The polymer scaffold of the invention support the transplanted cells and provides an optimal depot for these molecules. In the present invention, reporter molecules are incorporated into polymer scaffolds suitable for cell transplantation incorporating reporter molecules for cell behavior and assess the ability of this system to report on the cell behavior over time. This work will create a foundation for a whole new field of bioactive scaffolds that report on their cellular cargo following transplantation with the advantage of virually observing the reporter molecule and allowing for early response.

The term scaffold refers to a structure capable of supporting three-dimensional tissue formation. Cells are often implanted in or seeded on the scaffold material. Depending on the purpose of the scaffold, the structure is designed to allow cell attachment and migration, deliver and retain cells, biochemical factor and/or therapeutic factors, enable diffusion of cell nutrients and/or recombinant products into the organ and/or exert certain mechanical or biological influences to modify the surrounding cells. The scaffolds of the invention are loaded with reporter dyes that will allow for monitoring of the cell response of the transplanted cells and also the response of the surrounding endogenous cells.

The invention provides for scaffolds for transplantation comprising a sustained release reporter molecule, wherein the reporter molecule provides information on cell function or cell response after transplantation. The reporter molecule information may be provided by any reporter molecule that provides a response that is visible by eye such that the function or status of the transplanted cells may be monitored without invasive methods. The reporter molecules within the scaffold are fluorescent dye, fluorescent lipid, fluorescently labeled molecule, MRI contrast agent, PET agent or a chemiluminescent molecule. The invention may be carried out with any detectable reporter molecule and particularly those reporter molecules that are detectable by eye. Examples of commercially available florescent reporter molecules with a long track record in many cells include Ethidium Homodimer-1 (EthD-1) and Calcein AM (the Live/Dead assay molecules), CellROX® Green Reagent, and pHrodo™ Red AM, DAPI, FITC and Hoechst 33342.

The reporter molecule within the scaffolds of the invention may provide information on any cellular response, cellular behavior, the cellular environment or any cellular condition. For example, the reporter molecule may provide information on cell viability, apoptosis, oxidative stress, pH, cell cycle analysis, cell differentiation, activation states, protein localization, cell proliferation, expression of markers of inflammation, expression of complement-associated proteins, bacterial contamination, viral contamination, yeast contamination or fungal contamination.

In any of the scaffolds of the invention, the scaffold is a nanoparticle, film, nanofibers, dendrimers, hydrogel, or non-woven meshes of multiple polymers. In addition, any of the scaffolds of the invention may comprise a biodegradable polymer such as poly(lactic-co-glycolic acid (PLGA), polylactic acid (PLA), polyglycolic acid (PGA), poly (ε-caprolactone) (PCL), Poly-L-lysine (PLL), polyglycolide or combinations thereof. Alternatively, the scaffolds of the invention may comprise nonbiogradable materials or metals such as porous titanium fibers or mesh, ceramics such as hydroxyapatite, porcelain, bioglass, tricalcium phosphate, gold, silver, platinum, aluminum, palladium, copper, cobalt, indium, nickel, ZnS, ZnO, Ti, TiO₂, Sn, SnO₂, Si, SiO₂, Fe, Fe⁺⁴, steel, cobalt-chrome alloys, Cd, CdSe, CdS, and CdS, titanium alloy, AgI, AgBr, HgI₂, PbS, PbSe, ZnTe, CdTe, In₂S₃, In₂Se₃, Cd₃P₂, Cd₃As₂ InAs, or GaAs.

Any of the scaffolds of the invention are dissolved in a water miscible solvent, such as NMR. These scaffolds are used in a spray on system that when spread on, injected or applied to tissue, the scaffold polymer crashes our of solution and sticks to the tissue entrapping the reporter molecule.

In a particular embodiment, the scaffold is a nanoparticle comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein Ilb/Illa (GPIIb/IIIa). Optionally, the nanoparticle scaffold is dissolved in NMR, a water miscible solvent. For example, these nanoparticle scaffolds comprising a reporter molecule may be administered to subjects as synthetic platelets to alleviate bleeding and to facilitate wound healing. The invention also provides for use of a medicament to alleviate bleeding and to facilitate wound healing comprising the nanoparticle scaffolds of the invention.

In another embodiment, the scaffold is a PLGA film comprising a reporter molecule that is administered to the eye of a subject suffering from atrophic age-related macular degeneration (AMD). The invention also provides for use of a medicament to treat AMD comprising a scaffold of the invention, such as PLGA film scaffolds of the invention.

The reporter molecules are sustained released from the scaffold. The term “sustained released” refers to slowly releasing a molecule into the cells that were transplanted or into the surrounding cells or environment over an extended period of time. The data provided in Example 2 demonstrates that the reporter information continues to be detectable at least 4 weeks after implant or transplantation. The sustained release of the reporter molecule that continues to provide valuable information for the extended period of time is unexpected.

The extended period of time of the sustained release may be release spanning at least seven days (one week), spanning at least 10 days, spanning at least 12 days, spanning 14 days (two weeks), spanning at least 16 days, spanning at least 18 days, spanning at least 21 days (three weeks), spanning at least 25 days, spanning at least 28 days (four weeks), spanning at least 30 days, spanning at least 35 days (five weeks), spanning at least 38 days, spanning at least 42 days (six weeks), spanning at least 45 days, spanning at least 49 days (seven weeks), spanning at least 52 days, spanning at least 56 weeks (8 weeks), spanning at least 60 days, spanning at least 63 days (9 weeks), spanning at least 70 days (10 weeks), spanning at least 77 days (11 weeks) or spanning at least 84 days (12 weeks).

The sustained release period ranges from 1 to 2 weeks, or ranges from 1 to 4 weeks, or ranges from 1 to 6 weeks, or ranges from 1 to 8 weeks, or range from 2 to 3 weeks, or ranges from 2 to 4 weeks, or ranges from 2 to 6 weeks, or ranges from 2 to 8 weeks, or ranges from 2 to 10 weeks, or range from 3 to 4 weeks, or ranges from 3 to 6 weeks, or ranges from 3 to 8 weeks, or ranges from 3 to 8 weeks, or ranges from 3 to 10 weeks, or range from 4 to 5 weeks, or ranges 4 to 6 weeks, or ranges 4 to 8 weeks, or ranges 4 to 10 weeks, or ranges 4 to 12 weeks, or range from 5 to 6 weeks, or ranges from 5 to 8 weeks, or ranges from 5 to 10 weeks, or ranges from 5 to 12 weeks, or ranges from 6 to 7 weeks, or ranges from 6 to 8 weeks, or ranges from 6 to 10 weeks, or ranges from 6 to 12 weeks, or ranges from 7 to 8 weeks, or ranges from 7 to 10 weeks, or ranges from 7 to 12 weeks, or ranges from 8 to 9 weeks, or ranges from 8 to 10 weeks or ranges from 9 to 10 weeks, or ranges from 9 to 12 weeks or ranges from 10 to 11 weeks, or ranges from 10 to 12 weeks or ranges from 11 to 12 weeks.

The invention provides for methods of monitoring cell function or cell response after transplantation of a scaffold comprising the steps of a) administering a scaffold comprising a sustained release reporter molecule, wherein the reporter molecule provides information on cell function or cell response, b) detecting the reporter molecule in a cell in the transplanted scaffold or a cell in the endogenous tissue surrounding the scaffold, wherein detection of the reporter molecule provides information on cell function or cell response after transplantation.

In a further embodiment, the invention provides for methods of detecting infection after transplantation of a scaffold comprising the steps of a) administering a scaffold comprising a sustained release reporter molecule, wherein the reporter molecule provides information on infection and b) detecting the reporter molecule in a cell in the transplanted scaffold or a cell in the endogenous tissue surrounding the scaffold, wherein detection of the reporter molecule is indicative of infection. Markers of infection include C-reactive protein (CRP) and other markers of inflammation, complement associated proteins, presence of leukocytes, procalcitonin and expression of pro-inflammatory cytokines such as TNF-α, IL-1α, IL-4, IL-5, IL-6, IL-1β, IL-10, IL-12, IL-13, IL-17, IL-25, IFN-γ, and G-CSF.

The methods of the invention may be carried out with scaffold comprising any reporter molecules such as a fluorescent dye, fluorescent lipid, fluorescently labeled molecule, MRI contrast agent, PET agent or a chemiluminescent molecule. For example, any of the methods of the invention may be carried out with any detectable reporter molecule and particularly those reporter molecules that are visually detectable. Examples of commercially available florescent reporter molecules with a long track record in many cells include Ethidium Homodimer-1 (EthD-1) and Calcein AM (the Live/Dead assay molecules), CellROX® Green Reagent, and pHrodo™ Red AM.

The invention also provides for methods of detecting microorganism contamination of a scaffold comprising the steps of a) administering a scaffold comprising a sustained release reporter molecule, wherein the reporter molecule provides information on bacterial contamination, viral contamination, yeast contamination or fungal contamination and b) detecting the reporter molecule in a cell in the transplanted scaffold or a cell in the endogenous tissue surrounding the scaffold, wherein detection of the reporter molecule is indicative of microorganism organism.

The reporter molecule within the scaffolds administered by the methods of the invention may provide information on any cellular response, cellular behavior, the cellular environment or any cellular condition. For example, the reporter molecule may provide information on cell viability, apoptosis, oxidative stress, pH, cell cycle analysis, cell differentiation, activation states, protein localization, cell proliferation, expression of markers of inflammation, expression of complement-associated proteins, bacterial contamination, viral contamination, yeast contamination or fungal contamination.

In any of the methods of the invention, the scaffold administered is nanoparticles, film, nanofibers, dendrimers, hydrogel, or non-woven meshes of multiple polymers. In addition,in any of the methods of the invention, the scaffold comprise a biodegradable polymer such as PLGA, PLA, PGA,PCL, PLL, polyglycolide or combinations thereof. Alternatively, in any of the methods of the invention, the scaffolds administered may comprise nonbiogradable materials or metals such as porous titanium fibers or mesh, ceramics such as hydroxyapatite, porcelain and bioglass, and tricalcium phosphate, gold, silver, platinum, aluminum, palladium, copper, cobalt, indium, nickel, ZnS, ZnO, Ti, TiO₂, Sn, SnO₂, Si, SiO₂, Fe, Fe⁺⁴ , steel, cobalt-chrome alloys, Cd, CdSe, CdS, and CdS, titanium alloy, AgI, AgBr, HgI₂, PbS, PbSe, ZnTe, CdTe, In₂S₃, In₂Se₃, Cd₃P₂, Cd₃As₂, InAs, or GaAs.

In a particular, in any of the methods of the invention, the scaffold administered is a nanoparticle comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein IIb/IIIa (GPIIb/IIIa). Optionally the nanoparticle scaffold is dissolved in NMR, a water miscible solvent.

For example, the nanoparticle scaffolds comprising a reporter molecule may be administered to subjects as synthetic platelets to alleviate bleeding and to facilitate wound healing and any of the methods of the invention may be carried out to monitor the effect the synthetic platelets have on the surrounding cells, monitor the condition of the surrounding cells and/or to determine whether there is contamination or an infection.

In any of the methods of the invention, the scaffold is a PLGA film comprising a reporter molecule and the scaffold is administered to the eye of a subject suffering from AMD. Any of the methods of the invention may be carried out to monitor the effect of the scaffold on the surrounding cells, monitor the condition of the surrounding or transplanted and/or determine whether the surrounding cells or transplanted cells are contaminated or infected.

Another advantage of the present invention is that information on the function or status of the transplanted cells is available early after transplant.

### BRIEF DESCRIPTION OF DRAWING

FIG. 1 shows CellRox Green release data.

FIG. 2 shows the pHRodo Red release data.

FIG. 3 shows the effect of supernatant collected from CellRox Green containing scaffolds collected at 1 week.

FIG. 4 shows the effect of supernatant collected from CellRox Green containing scaffolds collected at 4 weeks.

FIG. 5 shows the effect of supernatant collected from pHRodo Red containing scaffolds collected at 1 week.

FIG. 6 shows the effect of supernatant collected from pHRodo Red containing scaffolds collected at 2 weeks.

### DETAILED DESCRIPTION

The present invention loads polymer scaffolds with sustained released report molecules and methods of using these scaffolds to monitor and observe the behavior and conditions of transplanted cells and the tissue surrounding the transplant. The report proteins are incorporated into scaffold, usually during fabrication, and will be released slowly over time. The reporter molecules allow for real time information which is visible to the eye. In addition, as this information is generated on the cellular level, the observer will have an opportunity to receive the information during an early phase of the condition, e.g. contamination or infection might be detectable before systemic markers such as inflammation or redness is observable.

The term scaffold refers to a structure capable of supporting three-dimensional tissue formation. Cells are often implanted in or seeded on the scaffold material. Depending on the purpose of the scaffold, the structure is designed to allow cell attachment and migration, deliver and retain cells, deliver biochemical factor and/or therapeutic factors, enable diffusion of cell nutrients and/or recombinant products into the organ and/or exert certain mechanical or biological influences to modify the surrounding cells. The scaffolds of the invention are loaded with reporter dyes that allow for monitoring of the response of the transplanted cells and also the response of the surrounding endogenous cells.

### Scaffolds

The invention may be carried out with any type of polymer scaffold including scaffolds comprising biodegradable polymers and, nonbiodegradable (permanent) polymers. For example, biodegradable scaffolds of the invention may comprise one or more biodegradable extracellular matrix proteins and polysaccharides such as collagen, fibrin, PuraMatrix, Matrigel, chitosan, glycosaminoglycans (GAGs) e.g. hyaluronic acid. The biodegradable scaffold may comprise a single polymer, a block copolymer, a triblock copolymer or a quadblock polymer such as PLGA, polylactic acid (PLA), polyglycolic acid (PGA), (poly(ε-caprolactone) PCL, PLL, polyglycolide or combinations thereof. The scaffold may be a combination of one or more extracellar matrix components and one or more polymers.

The scaffold may comprise a water soluble polymer such as water soluble polymer selected from the group consisting of polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammoniumphosphate (MPC), polyethylene glycol propionaldehyde, copolymers of ethylene glycol/propylene glycol, monomethoxy-polyethylene glycol, carboxymethylcellulose, polyacetals, poly-1,3-dioxolane, poly-1,3,6-trioxane, ethylene/maleic anhydride copolymer, poly (β-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and combinations or mixtures thereof.

Nondegradable or permanent scaffolds may comprise nondegradable metals such as porous titanium fibers or mesh, ceramics such as hydroxyapatite, porcelain and bioglass, and tricalcium phosphate. Such metal include gold, silver, platinum, aluminum, palladium, copper, cobalt, indium, nickel, ZnS, ZnO, Ti, TiO₂, Sn, Sn0₂, Si, SiO₂, Fe, Fe⁺⁴, steel, cobalt-chrome alloys, Cd, CdSe, CdS, and CdS, titanium alloy, AgI, AgBr, HgI₂, PbS, PbSe, ZnTe, CdTe, In₂S₃, In₂Se₃, Cd₃P₂, Cd₃As₂, InAs, GaAs

The invention may be carried out with any type of scaffold shape such as nanofibers, nanoparticles having a spheroid shape, nanoparticles having a non-spheroid shape such as rods, fiber and whiskers, dendrimers, hydrogels, films and non-woven meshes of multiple polymers. Example nanospheres may have a diameter of less than 1 micron such as a diameter between 0.1 micron and 1 micron.

Scaffolds of the disclosure are contemplated to have any of a number of different shapes. The shape of the scaffold is in certain aspects, a function of the method of its production. In other aspects, the scaffold acquires a shaped that is formed before, during or after the process of its production. For example, nanoparticle scaffolds are provided that have a spheroid shape. Spheroid nanoparticles (referred to herein as nanospheres) having various sizes are contemplated, wherein, for example nanoparticles having a diameter between 0.1 micron and 0.5 micron, between 0.2 micron and 0.4 micron, between 0.25 micron and 0.375 micron, between 0.3 micron and 0.375 micron, between 0.325 micron and 0.375 micron, between 0.12 microns and 0.22 microns, between 0.13 microns and 0.22 microns, between 0.14 microns and 0.22 microns, between 0.15 microns and 0.22 microns, between 0.16 microns and 0.22 microns, between 0.17 microns and 0.22 microns, between 0.18 microns and 0.22 microns, between 0.19 microns and 0.22 microns, between 0.20 microns and 0.22 microns, between 0.21 microns and 0.22 microns, between 0.12 microns and 0.21 microns, between 0.12 microns and 0.20 microns, between 0.12 microns and 0.19 microns, between 0.12 microns and 0.18 microns, between 0.12 microns and 0.17 microns, between 0.12 microns and 0.16 microns, between 0.12 microns and 0.15 microns, between 0.12 microns and 0.14 microns, or between 0.12 microns and 0.13 microns are contemplated. In various aspect, nanoparticles are contemplated having a diameter of 0.01 microns to 1.0 micron, 0.05 microns to 1.0 micron, 0.05 microns to 0.95 microns, 0.05 microns to 0.9 microns, 0.05 microns to 0.85 microns, 0.05 microns to 0.8 microns, 0.05 microns to 0.75 microns, 0.05 microns to 0.7 microns, 0.05 microns to 0.65 microns, 0.05 microns to 0.6 microns, 0.05 microns to 0.55 microns, 0.05 microns to 0.5 microns, 0.1 microns to 1 micron, 0.15 microns to 1.0 microns, 0.2 microns to 1 micron, 0.25 microns to 1.0 microns, 0.3 microns to 1 micron, 0.35 microns to 1.0 microns, 0.4 microns to 1 micron, 0.45 microns to 1.0 microns, or 0.5 microns to 1 micron. In compositions of nanoparticles provided by the disclosure, the spherical nanoparticles are homogenous in that that all have the same diameter, or they are heterogeneous in that at least two nanoparticles in the composition have different diameters.

Alternatively, in any of the compositions of the invention, the scaffold is a nanoparticles having a non-spheroid shape. For example, the nanoparticle is a rod, fiber or whisker. The nanoparticles may have an aspect ratio length to width of at least 3.

Examples of particular scaffolds known in the art at the time of filing include scaffolds: PLGA-based films, polylysine-poly(ethylenc glycol) hydrogel system, hemostatic nanoparticles (e.g. synthetic platelets).

The scaffold used in the present invention may comprise a therapeutic compound in addition to the reporting molecule. For example, the therapeutic compound is hydrophobic. The therapeutic compound is a growth factor, a cytokine, a steroid, or a small molecule or an anti-cancer compound. The invention provides for compositions which are pharmaceutical compositions comprising the scaffolds of the invention, wherein the composition further comprises a pharmaceutically acceptable carrier, diluent or formulation. For example, the compositions of the invention may be in an intravenous administration formulation. The compositions of the invention may be lyophilized or a powder.

The invention provides for methods of treating an condition in an individual comprising the step of administering any of the foregoing compositions to a patient in need thereof in an amount effective to treat the condition and the step of monitoring the effect of the therapeutic agent on the cells that the composition was administered to. For example, the invention provides for methods wherein the individual has a bleeding disorder and the composition is administered in an amount effective to reduce bleeding. In particular, the invention provide for methods of treating a bleeding disorder comprising the step of administering any of the foregoing compositions in an amount effective to reduce bleeding time by more than 15% compared to no administration or administration of saline. In these methods of the invention, the bleeding disorder may be a symptom of a clotting disorder, thrombocytopenia, a wound healing disorder, trauma, blast trauma, a spinal cord injury or hemorrhaging.

### Nanoparticles

An example of a nanoparticle scaffold is a nanoparticle comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein IIb/IIIa (GPIIb/IlIa). In various aspects, the peptide is linear or cyclic. It will be appreciated that in a composition comprising a plurality of nanoparticles of the disclosure, the composition is contemplated to include nanoparticles wherein all peptides are linear, all peptides are cyclic, or a mixture of linear and cyclic peptides is present.

Nanoparticle are also provided which are non-spheroid. Other nanoparticles include those having a rod, fiber or whisker shape. In rod, fiber or whisker embodiments, the nanoparticle has a sufficiently high aspect ratio to avoid, slow or reduce the rate of clearance from circulation.

Aspect ratio is a term understood in the art, a high aspect ratio indicates a long and narrow shape and a low aspect ratio indicates a short and thick shape.

Nanoparticles of the disclosure are contemplated with an aspect ratio length to width of at least 3, of at least 3.5, of at least 4.0, of at least 4.5, of at least 5.0, of at least 5.5, of at least 6.0, of at least 6.5, of at least 7.0, of at least 7.5, of at least 8.0, of at least 8.5, of at least 9.0, of at least 9.5, of at least 10.0 or more. In a composition of nanoparticles contemplated, the nanoparticles have, in one embodiment, identical aspect ratios, and in alternative embodiments, at least two nanoparticles in the composition have different aspects ratios. Composition of nanoparticles are also characterized by having, on average, essentially the same aspect ratio. “Essentially the same” as used in this instance indicated that variation in aspect ratio of about 10%, about 9%, about 8%, about 7% about 6% or up to about 5% is embraced. In still other aspects, a composition of nanoparticles is provided wherein the nanoparticles in the composition have an aspect ratio of between about 1% and 200%, between about 1% and 150%, between about 1% and 100%, between about 1% and about 50%, between about 50% and 200%, between about 100% and 200%, and between about 150% and 200%. Alternatively, the nanoparticles in the composition have an aspect ratio from about X % to Y %, wherein X from 1 up to 100 and Y is from 100 up to 200.

The disclosure further provides nanoparticles of essentially any shape are formed using microfabrication processes well known and routinely practiced in the art. In microfabrication methods, size and shape of the nanoparticles are predetermined by design.

The invention also provides for use of any of the nanoparticle scaffolds of the invention for the preparation of a medicament for the treatment of a condition wherein the medicament comprises the nanoparticle scaffolds in an amount effective to treat the condition. For example, the invention provides for a use of any of the foregoing nanoparticle scaffold of the invention for the preparation of a medicament for the treatment of a bleeding disorder wherein the medicament comprises the composition in an amount effective to reduce bleeding. The invention provides for an use of any of the foregoing nanoparticle scaffolds for the preparation of a medicament for the treatment of a bleeding disorder wherein the medicament comprise the composition in an mount effective to reduce bleeding time by more than 15% compared to no administration or administration of saline. In any of the uses of the invention, the medicament may be administered to treat a bleeding disorder that is a symptom of a clotting disorder, thrombocytopenia, a wound healing disorder, trauma, blast trauma, a spinal cord injury or hemorrhaging.

### Water Soluble Polymers

The scaffolds of the disclosure may comprise a water soluble polymer. Examples of water soluble polymers include polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(l-hydroxymethylethylene hydroxymethylformal) (PHF), 2-methacryloyloxy-2′-ethyltrimethylammoniumphosphate (MPC), polyethylene glycol propionaldehyde, copolymers of ethylene glycol/propylene glycol, monomethoxy-polyethylene glycol, carboxymethylcellulose, polyacetals, poly-1,3-dioxolane, poly-1,3,6-trioxane, ethylene/maleic anhydride copolymer, poly (β-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and combinations or mixtures thereof.

In a specific aspect, the scaffold of the disclosure comprises the water soluble polymer PEG. For nanoparticles in this aspect, the PEG has an average molecular weight between 100 Da and 10,000 Da, 500 Da and 10,000 Da, 1000 Da and 10,000 Da, 1500 Da and 10,000 Da, 2000 Da and 10,000 Da, 2500 Da and 10,000 Da, 3000 Da and 10,000 Da, 3500 Da and 10,000 Da, 4000 Da and 10,000 Da, 4500 Da and 10,000 Da, 5000 Da and 10,000 Da, 5500 Da and 10,000 Da, 1000 Da and 9500 Da, 1000 Da and 9000 Da, 1000 Da and 8500 Da, 1000 Da and 8000 Da, 1000 Da and 7500 Da, 1000 Da and 7000 Da, 1000 Da and 6500 Da, or 1000 Da and 6000 Da. Alternatively, the nanoparticle is one in which PEG has an average molecular weight of about 100, Da, 200 Da, 300 Da, 400 Da, 1000 Da, 1500 Da, 3000 Da, 3350 Da, 4000 Da, 4600 Da, 5,000 Da, 8,000 Da, or 10,000 Da.

The scaffolds of the disclosure include those comprising a water soluble polymer that is attached to the core at a molar ratio of 0.1:1, 0.2:1, 0.3:1, 0.4:1, 0.5:1, 0.6:1, 0.7:1, 0.8:1, 0.9:1, 1:1, 1:2, 1:3, 1:4, 1:5, 1:6, 1:7, 1:8, 1:9, 1:10 or greater. In various aspect, a plurality is provided wherein the water soluble polymer to core ratio is identical for each scaffold in the plurality, and in alternative aspect, at least two scaffolds in the plurality have different water soluble polymer to core ratios.

The degree to which a scaffold is associated with a water soluble polymer is, in various aspects, determined by the route of administration chosen.

### Reporter Molecules

The invention may be carried out with any detectable reporter molecule that reports on the status of cells or the cellular environment, particularly those reporter molecules that are visually detectable such as fluoroprobes, fluorescently labeled molecule, MRI contrast agent, PET agent or chemiluminescent agents. Examples of commercially available florescent reporter molecules with a long track record in many cells include Ethidium Homodimer-1 (EthD-1) and Calcein AM (the Live/Dead assay molecules), Cel1ROX® Green Reagent, pHrodo™ Red AM, Calcofluor White M2R for the staining of yeast, funcgo and bacterial contaminats, oxidative stress indicators, proliferation markers and markers of apoptosis. Other examples of fluoroprobes include Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Oregon-Green, Pacific Blue, Pacific Orange, Pacific Green, Cy3, Cy5, Coumarin, Fluorscein (FITC), Tetramethylrhodamine (TRITC), BODPY FL, and Texas Red.

The magnetic resonance imaging (MRI) contrast agents include gadolinium (Gd), iron oxide, iron platinum, manganese, protein-based MRI contrast agents. Positron emission tomography (PET) agents include diacetyl-bis(N⁴-methylthiosemicarbazone), also called ATSM or Copper 64, fluorodeoxyglucose (FDG) or other glucose analogs, fluoride, 3′-deoxy-3′-[¹⁸F]fluorothymidine (FLT), ¹⁸F-fluoromisonidazole, gallium, technetium-99M and Thallium

The reporter molecule may be a fluorescently labeled molecule such as a fluorescently labeled protein or peptide. For examples isothiocyanate derivatives, e.g. FITC and TRITC (derivatives of fluorescein and rhodamine) are reactive towards primary amines to form a thioureido linkage between the compound of interest and the dye. Succinimidyl esters such as NHS-fluorescein are reactive towards amino groups to form an amido bond. Maleimide activated fluorophores such as fluorescein-5-maleimide readily react with sulfhydryl groups. The sulfhydryl group adds to the double bond of the maleimide. Other protein labels include R-Phycoerythrin (R-PE) and Allophycocyanin (APC).

The reporter molecule within the scaffolds of the invention may provide information on any cellular response, cellular behavior, the cellular environment or any cellular condition. For example, the reporter molecule may provide information on cell viability, apoptosis, oxidative stress, pH, cell cycle analysis, cell differentiation, activation states, protein localization, cell proliferation, expression of markers of inflammation, expression of complement-associated proteins, bacterial contamination, viral contamination, yeast contamination or fungal contamination. Markers of inflammation include: C-reactive protein (CRP) and other markers of inflammation, complement associated proteins, presence of leukocytes, procalcitonin and expression of pro-inflammatory cytokines such as TNF-α, IL-1α, IL-4, IL-5, IL-6, IL-1β, IL-10, IL-12, IL-13, IL-17, IL-25, IFN-γ, and G-CSF.

The reporter molecules EthD-1 is cell impermeable in healthy cells and fluoresces red when bound to DNA. Calcein AM fluoresces green in live cells. CellROX® Green is a Molecular Probes fluorescent molecule based on fluoroescein that is green upon oxidation by reactive oxygen species (ROS). pHrodo™ Red AM is an intracellular pH Indicator based on rhodamine that is increasingly fluorescent as the pH drops from neutral. These molecules provide a strong foundation for developing reporter scaffolds. One can load cells with these molecules prior to transplantation, but they wash out quickly from the cells which gives only short-term data on the transplanted cells. Therefore, we propose to deliver these molecules from scaffolds used to support the transplanted cells.

### Therapeutic Agents

A scaffolds of the disclosure is also contemplated to further comprise a therapeutic compound. In various aspects, the therapeutic compound is hydrophobic and in still other aspects, the therapeutic compound is hydrophilic. A scaffold of the disclosure is provided wherein the therapeutic compound is covalently attached to thescaffold, non-covalently associated with the scaffold, associated with the scaffold through electrostatic interaction, or associated with the scaffold through hydrophobic interaction. In various embodiments, the therapeutic compound is a growth factor, a cytokine, a steroid, or a small molecule. Embodiments are contemplated wherein more than one therapeutic compound is associated with a scaffold. In this aspect, each therapeutic compounds associated with the scaffold is the same, or each therapeutic compound associated with the scaffold is different.

In various aspects, the therapeutic compound is an anti-cancer compound, and in specific embodiments, the therapeutic compound is selected from the group consisting of: an alkylating agents including without limitation nitrogen mustards, such as mechlor-ethamine, cyclophosphamide, ifosfamide, melphalan and chlorambucil; nitrosoureas, such as without limitation carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as without limitation busulfan; triazines such as dacarbazine (DTIC); antimetabolites including folic acid analogs such as methotrexate and trimetrexate; pyrimidine analogs such as without limitation 5-fluorouracil, fluorodeoxyuridine, gemcitabine, cytosine arabinoside (AraC, cytarabine), 5-azacytidine, 2,2′-difluorodeoxycytidine; purine analogs such as without limitation 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including without limitation antimitotic drugs such as paclitaxel; vinca alkaloids including without limitation vinblastine (VLB), vincristine, and vinorelbine, taxotere, estramustine, and estramustine phosphate; epipodophylotoxins such as without limitation etoposide and teniposide; antibiotics such as without limitation actimomycin D, daunomycin (rubidomycin), doxorubicin, mitoxantrone, idarubicin, bleomycins, plicamycin (mithramycin), mitomycinC, and actinomycin; enzymes such as without limitation L-asparaginase; biological response modifiers such as without limitation interferon-alpha, IL-2, G-CSF and GM-CSF; miscellaneous agents including without limitation platinum coordination complexes such as cisplatin and carboplatin; anthracenediones such as without limitation mitoxantrone; substituted urea such as without limitation hydroxyurea; methylhydrazine derivatives including without limitation N-methylhydrazine (MIH) and procarbazine; adrenocortical suppressants such as without limitation mitotane (o,p′-DDD) and aminoglutethimide; hormones and antagonists including without limitation adrenocorticosteroid antagonists such as prednisone and equivalents, dcxamcthasonc and aminoglutethimidc; progcstin such as without limitation hydroxyprogesterone caproate, medroxyprogesterone acetate and megestrol acetate; estrogen such as without limitation diethylstilbestrol and ethinyl estradiol equivalents; antiestrogen such as without limitation tamoxifen; androgens including testosterone propionate and fluoxymesterone/equivalents; antiandrogens such as without limitation flutamide, gonadotropin-releasing hormone analogs and leuprolide; non-steroidal antiandrogens such as without limitation flutamide; folate inhibitors; tyrosine kinase inhibitors such as without limitation AG1478, and radiosensitizing compounds.

In various aspects, the therapeutic compound is selected from the group consisting of AG1478, acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, droloxifene, dromostanolone, duazomycin, edatrexate, eflomithine, elsamitrucin, enloplatin, enpromate, epipropidine, epirubicin, erbulozole, esorubicin, estramustine, estramustine, etanidazole, etoposide, etoposide, etoprine, fadrozole, fazarabine, fenretinide, floxuridine, fludarabine, fluorouracil, flurocitabine, fosquidone, fostriecin, fulvestrant, gemcitabine, gemcitabine, hydroxyurea, idarubicin, ifosfamide, ilmofosine, interleukin II (IL-2, including recombinant interleukin II or rIL2), interferon alpha-2a, interferon alpha-2b, interferon alpha-n1, interferon alpha-n3, interferon beta-1a, interferon gamma-I b, iproplatin, irinotecan, lanreotide, letrozole, leuprolide, liarozole, lometrexol, lomustine, losoxantrone, masoprocol, maytansine, mechlorethamine hydrochlri de, megestrol, melengestrol acetate, melphalan, menogaril, mercaptopurine, methotrexate, methotrexate, metoprine, meturedepa, mitindomide, mitocarcin, mitocromin, mitogillin, mitomalcin, mitomycin, nitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, puromycin, pyrazofurin, riboprine, roglctimidc, safingol, safingol, semustine, simtrazcnc, sparfosatc, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, triethylenemelamine, trimetrexate, triptorelin, tubulozole, uracil mustard, uredepa, vapreotide, verteporlin, vinblastine, vincristine sulfate, vindesine, vinepidine, vinglycinate, vinleurosine, vinorelbine, vinrosidine, vinzolidine, vorozole, zeniplatin, zinostatin, zoledronate, and zorubicin. These and other antineoplastic therapeutic agents are described, for example, in Goodman & Gilman's The Pharmacological Basis of Therapeutics, McGraw-Hill Professional, 10th ed., 2001.

In various aspects, the therapeutic compound is an anti-inflammatory selected from the group consisting of glucocorticoids; kallikrein inhibitors; corticosteroids (e.g. without limitation, prednisone, methylprednisolone, dexamethasone, or triamcinalone acetinide); anti-inflammatory agents (such as without limitation noncorticosteroid anti-inflammatory compounds (e.g., without limitation ibuprofen or flubiproben)); vitamins and minerals (e.g., without limitation zinc); anti-oxidants (e.g., without limitation carotenoids (such as without limitation a xanthophyll carotenoid like zeaxanthin or lutein)) and agents that inhibit tumor necrosis factor (TNF) activity, such as without limitation adalimumab (HUMIRA®), infliximab REMICADE®), certolizumab (CIMZIA®), golimumab (SIMPONI®), and etanercept (ENBREL®).

In various aspects, the therapeutic compound is M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNF\ddot{γ}, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor \ddot{γ}, cytokine-induced eutrophils chemotactic factor 1, cytokine-induced eutrophils, chemotactic factor 2, cytokine-induced neutrophils chemotactic factor 2, endothelial cell growth factor, endothelin 1, epithelial-derived cutrophils attractant, glial cell line-derived neutrophic factor receptor 1, glial cell line-derived neutrophic factor receptor 2, growth related protein, growth related protein, growth related protein \ddot{γ}, growth related protein, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor, nerve growth factor nerve growth factor receptor, neurotrophin-3, neurotrophin-4, pre-B cell growth stimulating factor, stem cell factor, stem cell factor receptor, transforming growth factor, transforming growth factor, transforming growth factor, transforming growth factor 2, transforming growth factor \ddot{γ}, transforming growth factor, transforming growth factor β, latent transforming growth factor β, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, intracellular sigma peptide (ISP), and chimeric proteins and biologically or immunologically active fragments thereof.

Methods are also provided for with anticoagulation drugs. Including, for example and without limitation, plavix, aspirin, warfarin, heparin, ticlopidine, enoxaparin, Coumadin, dicumarol, acenocoumarol, citric acid, lepirudin and combinations thereof.

Methods in this aspects overcome the effects of these anticoagulant drugs which would be extremely helpful in surgery.

### Pharmaceutical Compositions

The invention provides a pharmaceutical composition comprising a scaffold of the disclosure. In various aspects, the pharmaceutical composition is a unit dose formulation. In various aspects, the pharmaceutical composition is an intravenous administration formulation. In various aspects, the pharmaceutical composition is lyophilized or a powder. In various aspects the pharmaceutical composition further comprises polyacrylic acid, poloxamer 188 or PEG.

The composition of the invention may be formulated for intravenous administration. The compositions may be delivered intravenously through infusion or injection, through a catheter, central line, tunneled line or through an implantable port.

In various aspects, a topical formulation is provided. Internal and external uses are provided wherein. The pharmaceutical composition for topical administration optionally includes a carrier, and is formulated as a solution, emulsion, ointment or gel base. The base, for example, optionally comprises one or more of the following: petrolatum, lanolin, polyethylene glycols, beeswax, mineral oil, diluents such as water and alcohol, and emulsifiers and stabilizers. Thickening agents are optionally present in a pharmaceutical composition for topical administration. In certain aspects, a solvent is in the formulation, the solvent including for example and without limitation, dimethyl sulfoxide (DMSO), NMP (N-Methyl-2-pyrrolidone), or a similar compound.

The compositions of the invention may be formulated for administration using a spray-on system. In one exemplary spray system, the scaffolds within the composition may or may not be suspended or dissolved in a carrier such as water. In another spray system, the scaffolds within the compositions are suspended or dissolved at various ratios in a water miscible such as DMSO, NMP, dimethylformamide (DMF) or tetrahydrofuran (THF). The compositions are then administered directly on the internal or external site of injury using a spray system, a brush system or syringe-type system. The spray system may be a wet or dry aerosol spray or wet or dry electrostatic spray. Alternatively, these compositions may be introduced to the injury using an endoscopic or other laproscopic device.

In other aspects, the compositions may be formulated for an oral, subcutaneous, intramuscular, transdermal, transbuccal, parenteral or sublingual route.

The invention provides pharmaceutical compositions formulated for delivery of scaffolds at 1 mg/kg to 1 g/kg, 10 mg/kg to 1 g/kg, 20 mg/kg to 1 g/kg, 30 mg/kg to 1 g/kg, 40 mg/kg to 1 g/kg, 50 mg/kg to 1 g/kg, 60 mg/kg to 1 g/kg, 70 mg/kg to 1 g/kg, 80 mg/kg to 1 g/kg, 90 mg/kg to 1 g/kg, 10 mg/kg to 900 mg/kg, 10 mg/kg to 800 m/kg, 10 mg/kg to 700 mg/kg, 10 mg/kg to 600 mg/kg, 10 mg/kg to 500 mg/kg, 10 mg/kg to 400 mg/kg, 10 mg/kg to 300 mg/kg, 10 mg/kg to 200 mg/kg, 10 mg/kg to 100 mg/kg, 10 mg/kg to 75 mg/kg, 10 mg/kg to 50 mg/kg, 50 mg/kg to 900 mg/kg, 100 mg/kg to 800 mg/kg, 200 mg/kg to 700 mg/kg, 300 mg/kg to 600 mg/kg, 400 mg/kg to 500 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, 300 mg/kg, 400 mg/kg, 500 mg/kg, 600 mg/kg, 700 mg/kg, 800 mg/kg, 900 mg/kg, 1000 mg/kg, or more.

Single dose administrations are provided, as well as multiple dose administrations. Multiple dose administration includes those wherein a second dose is administered within minutes, hours, day, weeks, or months after an initial administration.

### Uses of the Compositions

A method of treating a condition in an individual is provided comprising the step of administering a scaffold of the invention to a patient in need thereof in an amount effective to treat the condition, and then monitoring the effect of the scaffold on the cellular environment or the status, behavior or condition of the cells surrounding or within the scaffold.

One exemplary use of the invention is administering a scaffold, such as a nanoparticle scaffold or synthetic platelet, to an individual having a bleeding disorder. Methods are provided wherein the scaffold is administered in an amount effective to reduce bleeding time by more than 15%, by more than 20%, by more than 25%, or by more than 30% compared to no administration or administration of saline. In various aspects, the method is used wherein the bleeding disorder is a symptom of a clotting disorder, an acquired platelet function defect, a congenital platelet function defect, a congenital protein C or S deficiency, disseminated intravascular coagulation (DIC), Factor II deficiency, Factor V deficiency, Factor VII deficiency, Factor X deficiency, Factor XII deficiency, Hemophilia A, Hemophilia B, Idiopathic thrombocytopenic purpura (ITP), von Willebrand's disease (types I, II, and III), megakaryocyte/platelet deficiency. In various aspects, a method is provided wherein the condition is thrombocytopenia arising from chemotherapy and other therapy with a variety of drugs, radiation therapy, surgery, accidental blood loss, and other specific disease conditions. In various aspects, a method is provided wherein the condition is aplastic anemia, idiopathic or immune thrombocytopenia (ITP), including idiopathic thrombocytopenic purpura associated with breast cancer metastatic tumors which result in thrombocytopenia, systemic lupus erythematosus, including neonatal lupus syndrome, metastatic tumors which result in thrombocytopenia, splenomegaly, Fanconi's syndrome, vitamin B12 deficiency, folic acid deficiency, May-Hegglin anomaly, Wiskott-Aldrich syndrome, paroxysmal nocturnal hemoglobinuria, HIV associated ITP and HIV-related thrombotic thrombocytopenic purpura; chronic liver disease; myelodysplastic syndrome associated with thrombocytopenia; paroxysmal nocturnal hemoglobinuria, acute profound thrombocytopenia following C7E3 Fab (Abciximab) therapy; alloimmune thrombocytopenia, including maternal alloimmune thrombocytopenia; thrombocytopenia associated with antiphospholipid antibodies and thrombosis; autoimmune thrombocytopenia; drug-induced immune thrombocytopenia, including carboplatin-induced thrombocytopenia, heparin-induced thrombocytopenia; fetal thrombocytopenia; gestational thrombocytopenia; Hughes' syndrome; lupoid thrombocytopenia; accidental and/or massive blood loss; myeloproliferative disorders; thrombocytopenia in patients with malignancies; thrombotic thrombocytopenia purpura, including thrombotic microangiopathy manifesting as thrombotic thrombocytopenic purpura/hemolytic uremic syndrome in cancer patients; autoimmune hemolytic anemia; occult jejunal diverticulum perforation; pure red cell aplasia; autoimmune thrombocytopenia; nephropathia epidemica; rifampicin-associated acute renal failure; Paris-Trousseau thrombocytopenia; neonatal alloimmune thrombocytopenia; paroxysmal nocturnal hemoglobinuria; hematologic changes in stomach cancer; hemolytic uremic syndromes in childhood; and hematologic manifestations related to viral infection including hepatitis A virus and CMV-associated thrombocytopenia. In various aspects, a method is provided wherein the condition arises from treatment for AIDS which result in thrombocytopenia. In various aspects, the treatment for AIDS is administration of AZT.

In various aspect, the individual being treated is suffering from a wound healing disorders, trauma, blast trauma, a spinal cord injury, hemorrhagic stroke, hemorrhaging following administration of TPA, or intraventricular hemorrhaging which is seen in many conditions but especially acute in premature births.

Other aspects and advantages of the present invention will be understood upon consideration of the following illustrative examples.

### EXAMPLES

### Example 1

### Fabricating Scaffolds Incorporating Reporter Molecules

Reporter molecules are encapsulated into a scaffold alone or in pairs (e.g. live/dead pair and the oxidative stress/pH pair), such as in PLGA-based films or synthetic platelets. The encapsulation (or loading) of the reporter molecules will most often occur at the time of fabrication of the scaffold. To measure release of the reporter molecule, the scaffolds are incubated in PBS at 37° C. and the supernatant is collected over time to generate release curves for the reporter molecules.

The supernatants are administered to 3T3 fibroblasts to determine the bioactivity of the reporter molecules on normal cells and cells under stress. Stress is induced by incubating the cells with different percentages of ethanol prior to incubation with the released reporter molecules.

Alternatively, the 3T3 fibroblasts are seeded on the scaffolds and the efficacy of the scaffolds in reporting on the cell behavior over time from one to 21 days post culture is determined.

### Example 2

### PLGA-Based Film for Treating Atrophic Age-Related Macular Degeneration (AMD)

Reporter scaffolds based on degradable polymer scaffolds loaded with fluorescent reporter dyes for oxidative stress and intracellular pH as model reporters were generated In vivo, these dyes wash out of cells quickly. By incorporating the dyes directly into the scaffolds, cellular behavior can be observed not just over hours or days but over weeks and potentially months or longer.

CellROX® Green is a Molecular Probes fluorescent molecule based on fluoroescein that is green upon oxidation by reactive oxygen species (ROS). pHrodo™ Red AM is an intracellular pH Indicator based on rhodamine that is increasingly fluorescent as the pH drops from neutral.

PLGA 502H films were loaded with the Ce11ROX Green molecule probe or pHrodo Red Am. During fabrication, 4 ml of 10% polymer solution (PLGA 502H in CHC1₃) was mixed with 50 μl Ce11ROX Green or 4 ul pHRodo Red.

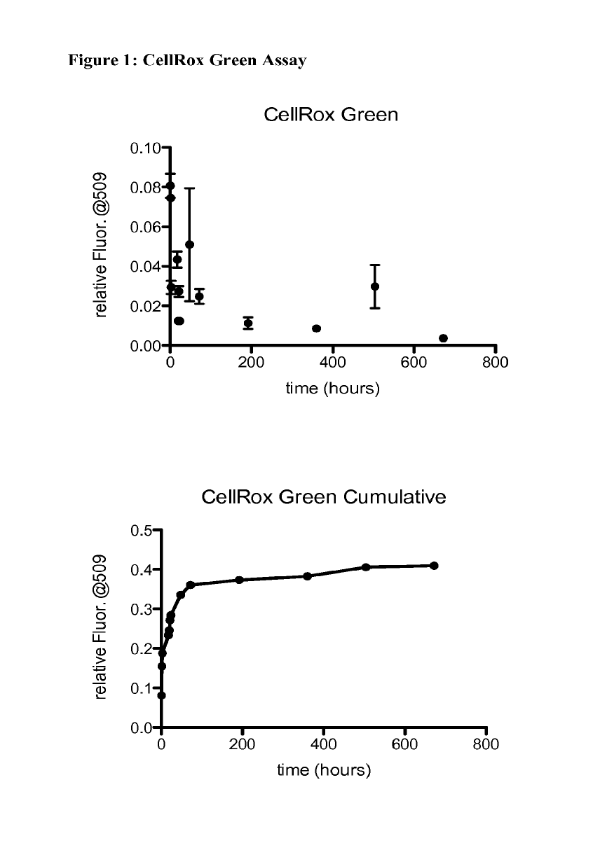

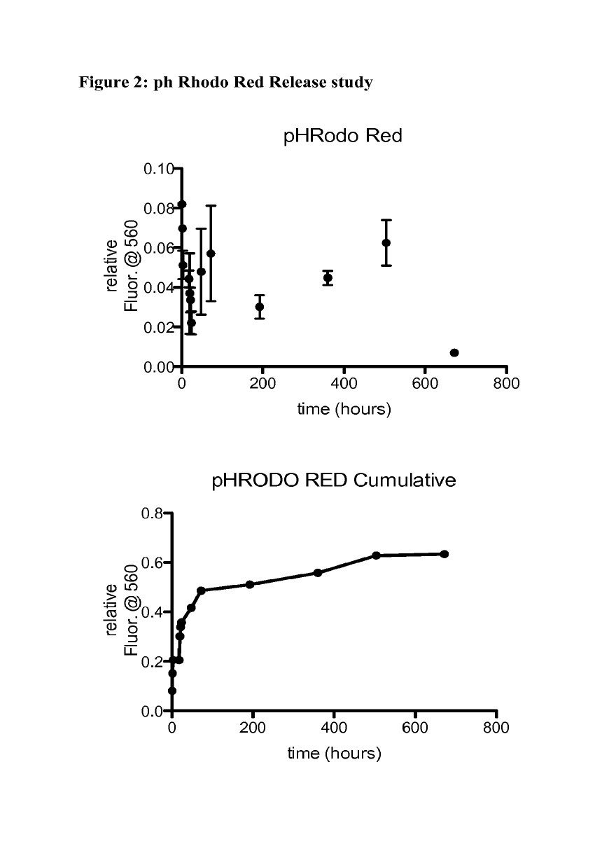

To investigate release of the reporter molecules, 120 mg films were incubated in 1 ml PBS at 37° C. under infinite sink conditions in triplicate. The data measuring CellRox Green Release is presented in FIG. 1 and the data measuring pHRodo Red Release is present in FIG. 2.

To investigate whether the reporter molecules provided information on the cellular environment after release from the scaffold, supernatant collected from the scaffold cultures at specific release time points was incubated with the rat endothelial cells that were known to be under oxidative stress (grown in the presence of alcohol).

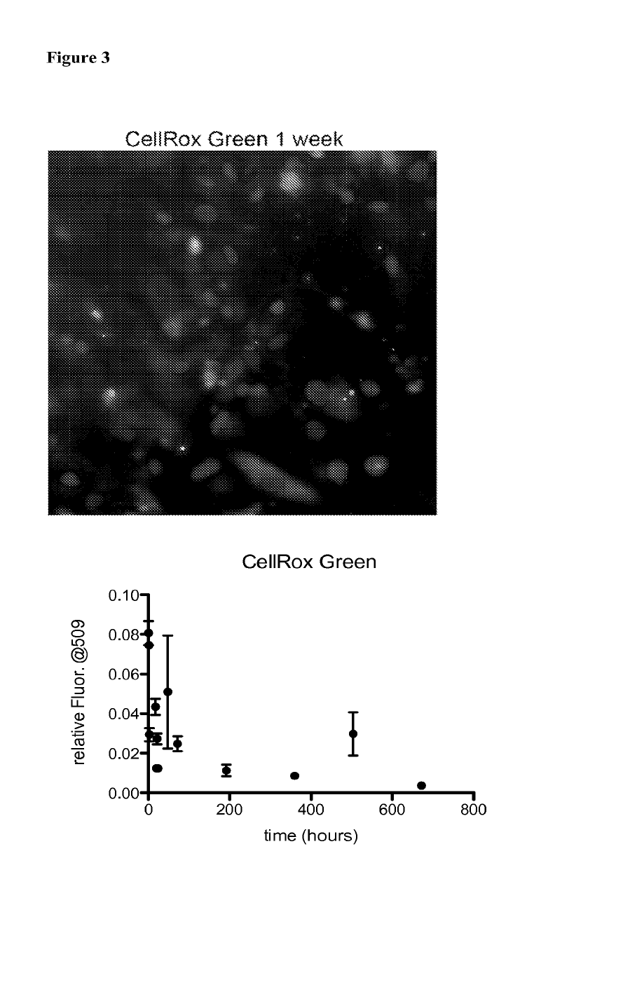

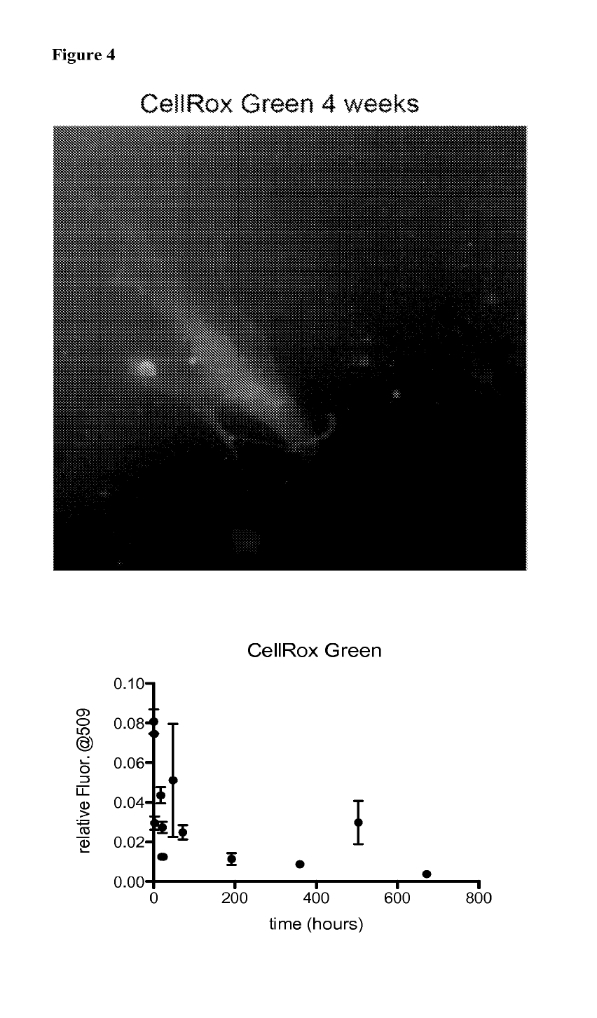

The supernatants from the scaffolds comprising CellROX green were collected after 200 hours (about 1 week) and after 700 hours (4 weeks) and were incubated with the endothelial cells under oxidative stress. The CellROX green in the supernatant were taken up by the endothelial cells and reported that the cells were under oxidative stress as shown in FIG. 3 (1 week supernatant) and FIG. 4 (4 week supernatant) by the presence of the fluorescent green color. Importantly, even the amount released at 4 weeks (FIG. 3), the smallest amount collected, led to positive reporting, comparable to the control cells incubated with fresh CellROX Green.

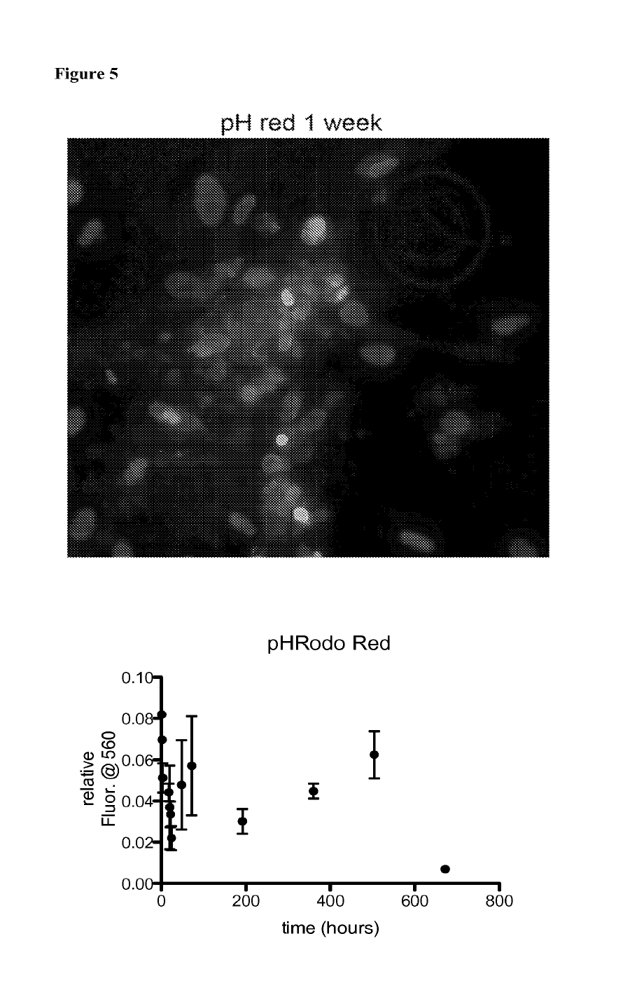

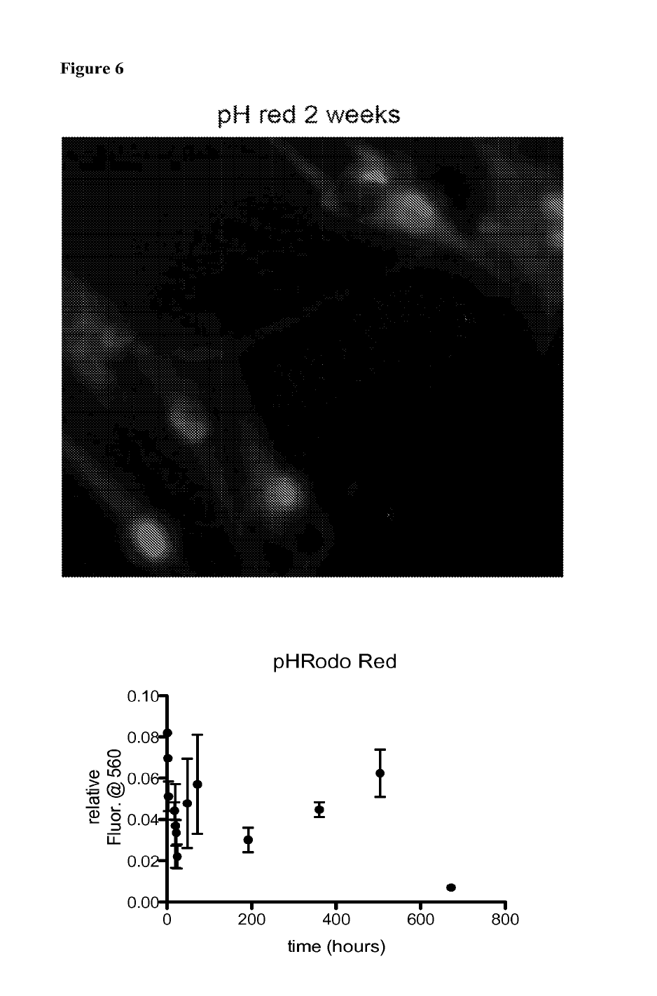

The supernatants from the scaffolds comprising pHRodo red were collected after 200 hours (about 1 week) and after 400 hours (2 weeks) and were incubated with the endothelial cells. The pHRodo red in the supernatant were taken up by the endothelial cells and reported that the cells had an acidic intracellular pH as they were under oxidative stress as shown in FIG. 5 (1 week supernatant) and FIG. 6 (2 week supernatant).

These PLGA films may be administered in vivo in the eye to treat age-related macular degeneration (AMD) and the release of the reporter molecule allows for the monitoring of the cellular behavior of the transplanted cell over time.

### Example

### 3

### Incorporation of Reporter Approach into Hemostatic Nanoparticles

During the fabrication process for the synthetic platelets, fluorophores are added to the system. An example of a procedure for making synthetic platelets that incorporates a fluorophore is below:

PLGA-PLL-PEG (1 g) is dissolved in anhydrous DMSO to a concentration of 100 mg/ml. Oligopeptides (25 mg GRGDS or GRADSP) is dissolved in 1 ml DMSO and added to the stirring polymer solution. This is reacted for 3 hours, and then transferred to dialysis tubing (SpectraPor 2 kDa MWCO). Dialysis water is changed every half hour for 4 hours with Type I D.I. water. The product is then snap-frozen in liquid nitrogen and lyophilized for 2-5 days.

The resulting quadblock copolymer PLGA-PLL-PEG-GRGDS (or a blend with PLGA-PLL-PEG) is then dissolved to a concentration of 20 mg/ml in acetonitrile (120 mg/6 ml). The fluorophore is added to the organic solution. This solution is added dropwise to a stirring volume of PBS. Precipitated nanoparticles form as the water-miscible solvent dissipates. Particles are collected using a coacervate precipitation method. Briefly, one mass equivalent of dry poly(acrylic acid) is added to the stirring particle suspension. 15 ml of 1% w/v pAA is then added slowly to the stirring suspension until flocculation occurs. After 5 minutes, the flocculated particles are collected by centrifugation at 500 g, and rinsed 3 times with 1% pAA (centrifuging at 500 g, 2 m, 4C between rinses). On the final rinse, particles are resuspended with D.I. water, snap-frozen and lyophilized for 2-5 days.

### Example 4

### Incorporation of Reporter Approach Into Spray on Scaffold

The polymer (PLGA-PLL-PEG-peptide, PLGA, or PLGA-PEG as examples) is dissolved in NMR, a water miscible solvent. The reporter molecule is also dissolved in this solution. When the solution is sprayed on the wound, the polymer crashes out of solution, entrapping the reporter molecule in the film. This, then, leaves a film on the wound that slowly delivers the reporter molecule of interest such as a reporter on whether the wound has bacterial or fungal contamination.

### Example 5

### Incorporation of Reporter Approach into Degradable Polymer Scaffolds

The reporter molecule is dissolved along with the degradable polymer in the organic solvent of interest such as chloroform. The solution is then cast over salt or electrosprayed to create a scaffold. The process of creating the scaffold entraps the reporter molecule inside such that it is delivered over days, weeks, or months following the culture of the scaffold with cells or implantation of the scaffold in vivo.

### Example 6

### Freeze-Dried Scaffolds

In this example, the reporter molecule is dissolved in dioxane along with the polymer. The solution is then frozen and the dioxane is sublimated under vacuum leaving a porous scaffold. As in previous examples, the reporter molecule is slowly released from the scaffold and taken up by the surrounding cells/environment in vitro or in vivo over days, weeks, or months.

The rate of release can be tailored by tailoring the degradation of the scaffold via the choice of polymer.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

1. A scaffold for transplantation comprising a sustained release reporter molecule, wherein the reporter molecule provides information on cell function or cell response after transplantation.

2. The scaffold of claim 1 wherein the reporter molecule is a fluorescent dye, fluorescent lipid, fluorescently labeled molecules, MRI agent, PET agent or a chemiluminescent molecule.

3. The scaffold of claim 1 wherein the reporter molecule provides information on cell viability, apoptosis, oxidative stress, cell cycle analysis, cell differentiation, activation states, protein localization, pH, cell proliferation, expression of markers of inflammation, expression of complement-associated proteins, bacterial contamination, viral contamination, yeast contamination or fungal contamination.

4. The scaffold of claim 1, wherein the scaffold is a nanoparticle, film, nanofibers, dendrimers, hydrogels, or non-woven meshes of multiple polymers.

5. The scaffold of claim 1, wherein the scaffold comprises a biodegradable polymer.

6. The scaffold of claim 5, wherein the biodegradable polymer is poly(lactic-co-glycolic acid PLGA, polylactic acid (PLA), polyglycolic acid (PGA), poly (ε-caprolactone) (PCL), poly-L-lysine (PLL), polyglycolide or combinations thereof.

7. The scaffold of claim 1, wherein the scaffold is a nanoparticle comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein IIb/IIIa (GPIIb/IIIa).

8. The scaffold of claim 1, wherein the scaffold is dissolved in a water miscible solvent.

9. A method of monitoring cell function or cell response after transplantation of a scaffold comprising the steps of
a) administering a scaffold comprising a sustained release reporter molecule, wherein the reporter molecule provides information on cell function or cell response,
b) detecting the reporter molecule in a cell in the transplanted scaffold or a cell in the endogenous tissue surrounding the scaffold, wherein detection of the reporter molecule provides information on cell function or cell response after transplantation.

10. The method of claim 9 wherein the reporter molecule is a fluorescent dye, fluorescent lipid, fluorescently labeled molecule, MRI contrast agent, PET agent or a chemiluminescent molecule.

11. The method of claim 9 wherein the reporter molecule provides information of cell viability, apoptosis, oxidative stress, cell cycle analysis, cell differentiation, activation states, protein localization, pH. cell proliferation, expression of markers of inflammation, expression of complement-associated proteins, bacterial contamination, viral contamination, yeast contamination or fungal contamination.

12. The method of claim 9, wherein the scaffold is a nanoparticle, film, nanofibers, dendrimers, hydrogels, or non-woven meshes of multiple polymers.

13. The method of claim 9, wherein the scaffold comprises a biodegradable polymer.

14. The method of claim 13, wherein the biodegradable polymer is poly(lactic-co-glycolic acid (PLGA), polylactic acid (PLA), polyglycolic acid (PGA), poly (ε-caprolactone) (PCL), poly-L-lysine (PLL), polyglycolide or combinations thereof.

15. The method of claim 9, wherein the scaffold is dissolved in a water miscible solvent and administered using a spray system.

16. The method of claim 9, wherein the scaffold is a nanoparticle comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein IIb/IIIa (GPIIb/IIIa).

17. A method of detecting infection after transplantation of a scaffold comprising the steps of
a) administering a scaffold comprising a sustained release reporter molecule, wherein the reporter molecule provides information on infection and
b) detecting the reporter molecule in a cell in the transplanted scaffold or a cell in the endogenous tissue surrounding the scaffold, wherein detection of the reporter molecule is indicative of infection.

18. The method of claim 17 wherein the reporter molecule is a fluorescent dye, fluorescent lipid, fluorescently labeled molecule, MRI contrast agent, PET agent or a chemiluminescent molecule.

19. The method of claim 17 wherein the reporter molecule provides information on expression of markers of inflammation or expression of complement-associated proteins.

20. The method of claim 17 wherein the reporter molecule indicates the presence of bacterial contamination, viral contamination, yeast contamination or fungal contamination.

21. The method of claim 17, wherein the scaffold is a nanoparticle, film, nanofibers, dendrimers, hydrogels, or non-woven meshes of multiple polymers.

22. The method of claim 17, wherein the scaffold comprises a biodegradable polymer.

23. The method of claim 22 wherein the biodegradable polymer PLGA, polylactic acid (PLA), polyglycolic acid (PGA), (poly (ε-caprolactone) PCL, PLL, polyglycolide or combinations thereof.

24. The method of claim 17, wherein the scaffold is dissolved in a water miscible solvent and administered using a spray system.

25. The method of claim 17, wherein the scaffold is a nanoparticle comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein IIb/IIIa (GPIIb/IIIa).
